(19) United States
(12) Patent Application Publication
(10) Pub. No.: US 2010/0021563 A1
(43) Pub. Date: Jan. 28, 2010

(54) COMPOSITIONS COMPRISING COCONUT OIL AND METHODS OF USE THEREOF

(76) Inventors: Paul LEVESQUE, Freeport, ME (US); Matthew Buderer, Oak Harbor, OH (US); George Roentsch, Stoddard, NH (US); Eugene Gresh, Elington, CT (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/509,014

(22) Filed: Jul. 24, 2009

Related U.S. Application Data
(60) Provisional application No. 61/083,246, filed on Jul. 24, 2008.

(51) Int. Cl. A61K 33/30; A61K 36/889; A61P 31/00

(52) U.S. Cl. 424/642; 424/727

(57) ABSTRACT

The present disclosure relates to natural and/or homeopathic compositions comprising coconut oil in a form suitable for topical administration to a subject and having one or more health-promoting property, including those for treatment and/or prevention of disease, and methods for use thereof. The present disclosure further relates to natural and/or homeopathic compositions comprising coconut oil and optionally at least one additional active agent, which may impart natural, homeopathic, medicinal, pharmaceutical, and/or cosmetic properties, and methods for use thereof.

This application claims priority to U.S. Provisional Patent Application No. 61/083,246, filed on Jul. 24, 2008, the entire disclosure of which is incorporated by reference herein.

The present disclosure relates generally to compositions comprising coconut oil as an active agent. More specifically, the present disclosure relates to natural and/or homeopathic products comprising coconut oil in a form suitable for topical administration to a subject and having one or more health-promoting property, including those for treatment and/or prevention of disease. The compositions presently disclosed may have antimicrobial properties, particularly antiviral properties. The present disclosure further relates to methods of using these compositions to promote good health and/or for treatment and/or prevention of disease.

### BACKGROUND

Coconut oil is a natural source of medium chain fatty acids and is typically extracted directly from coconut meat and separated from the water content. This “virgin” or “unrefined” coconut oil has a melting point that may range from 76° F. to 92° F. and is also known generally as “coconut oil 76” and “coconut oil 92.” “Fractionated” coconut oil refers to coconut oil that has been refined, for example, to comprise only the saturated fat portion of the oil by removing the long-chain triglycerides. Coconut oil is remarkably stable, slow to oxidize, and known to have a long shelf-life.

Coconut oil is recognized in the art as a valuable natural product that promotes health in a variety of ways. In addition to its nutritional value, coconut oil is useful as skin emollient and is also known to have antimicrobial properties, particularly antiviral properties. Kabara (The Pharmacological Effect of Lipids, The American Oil Chemists' Society, 1978) was among the first to report that fatty acids and their derivatives can inactivate certain microbes by damaging their lipid membranes, thereby precluding replication. Antimicrobial activity of coconut oil has been reported (Dayrit, XXXVII Cocotech Meeting, Chennai, India, Jul. 25, 2000), as well as that of various individual fatty acids that comprise coconut oil. For example, lauric acid (C₁₂), which comprises nearly 50% of coconut oil, and capric acid (C₁₀), which contributes to about another 10% of coconut oil, have both shown activity towards viruses and pathogenic bacteria either in the free form or their monoglyceride derivatives (monolaurin and monocaprin, respectively). In particular, lauric acid and/or monolaurin have shown activity toward HIV, herpes virus, Junin virus, vesicular stomatitis virus, cytomegalovirus, and influenza (Bartolotta et al., Arch Virol, 146, 777-790, 2001; Hornung et al., J Gen Virol, 75, 353-361, 1994; Kristmundsdóttir et al., J Pharm Sci, 88, 1011-1015, 1999; Enig, 1999), as well as toward pathogenic bacteria including Escherichia coli, Staphylococcus aureus, Listeria monocytogenes, and Helicobacter pylori (McLay et al., Int J Food Microbiol, 73, 1-9, 2002; Enig, AVOC Lauric Oils Symposium, Ho Chi Min City, Vietnam, 25 Apr. 1996). Caprylic acid (C₈) is another fatty acid that comprises coconut oil and has demonstrated health benefits, including antiviral activity.

The present inventors have found that due to its combination of emollient and antimicrobial properties, particularly antiviral and antibiotic properties, coconut oil may be useful as a barrier against disease. Moreover, the present inventors have found that the health benefits of coconut oil may be combined with other active ingredients, including, but not limited to, active agents comprising trace minerals, such as zinc, manganese, and selenium.

Zinc is a trace mineral that is also known in the art to provide certain health benefits. Elemental or ionic zinc has been shown to be effective in humans for the treatment of the common cold caused by the rhinovirus. Absorption of zinc into the body requires a salt form such as, but not limited to, zinc acetate, zinc chloride, zinc gluconate, zinc oxide, and zinc sulfate. Zinc is thought to combine with carboxyl sites on the rhinovirus, which prevents the virus from binding to intercellular adhesion molecules (ICAM-1) in the respiratory tract. (Mossad, Q J Med, 96, 35-43, 2003; Hulisz, J Am Pharm Assoc, 44, 594-603, 2004). Studies show that for greatest effectiveness, zinc should be administered in the elemental or ionic form and within 24 hours of the onset of symptoms. Patients treated with zinc have reported a reduction in the severity and duration of cold-like symptoms including sore throat, nasal congestion, nasal drainage, mucus secretion, coughing, hoarseness, sneezing, headache, muscle ache, and fever.

The art describes various zinc formulations that have been explored to introduce elemental or ionic zinc into the body, including administration of salt forms. Several formulations exist for oral administration; however this route has several drawbacks. Release of zinc in the oral tract can delay, or even prevent, the proper dose from reaching the respiratory system. Oral lozenges of zinc are also associated with undesirable side effects, including bad taste and nausea.

Because the main antiviral activity is thought to occur in the nasal passages, application of zinc directly to the nasal cavity has been preferred over oral administration in recent years. U.S. Pat. Nos. 7,348,360 and 7,115,275 and U.S. Patent Application Publication Nos. 2006/0275343 and 2007/0265337 disclose a zinc gluconate gel for application to the nasal cavity. This gel suffers drawbacks due to slow permeation of the nasal membrane where it can require four or more hours to dissipate, and has also been reported to cause a loss of smell.

Thus, to promote good health and for treatment and/or prevention of disease, there remains a need in the art for compositions comprising coconut oil that may be applied to the body to promote health and/or for treatment and/or prevention of disease. Further, there remains a need to combine the health benefits of coconut oil with other active agents. In particular, despite the formulations known in the art and discussed herein, there remains a need for a composition capable of delivering a dose of elemental or ionic zinc to the body in an amount effective to reduce at least one symptom associated with the common cold. The present disclosure addresses at least these needs.

### SUMMARY OF THE INVENTION

Generally, the present disclosure provides a composition comprising coconut oil that is in a form suitable for topical administration. More specifically, the present disclosure provides natural and/or homeopathic compositions comprising coconut oil as an active agent, wherein the composition is in a form suitable for topical application.

The present disclosure further relates to natural and/or homeopathic compositions comprising coconut oil as an active agent and at least one additional active agent having natural, homeopathic, medicinal, pharmaceutical, and/or cosmetic properties, wherein the composition is in a form suitable for topical application. More specifically, the present disclosure relates to compositions comprising zinc as at least one additional active agent.

The present disclosure further relates to methods of using these compositions to promote good health and/or for treatment and/or prevention of microbial infections, particularly viral infections, and/or disease. Additional characteristics and advantages of the compositions presently disclosed are described below.

It is understood that the foregoing description is purely exemplary and explanatory, and non-limiting of the full scope of the invention, as claimed.

Definitions

The term “antimicrobial,” as used herein, refers to the ability of an agent to kill or otherwise inhibit the growth of microbes including, but not limited to, viruses, bacteria, fungi, protozoa, and combinations thereof. The term “antiviral” as used herein, refers to the ability of an agent to kill or otherwise inhibit the growth of viruses. The term “antibiotic”, as used herein, means the ability of an agent to kill or otherwise inhibit the growth of bacteria.

“Fractionated coconut oil”, as used herein, refers to coconut oil that has been refined to remove long-chain glycerides.

“Virgin” and “unrefined” coconut oil, “coconut oil 76,” and “coconut oil 92” as used herein, refer to coconut oil in its natural state, as extracted from coconut meat and separated from the water content.

“Coconut oil” as used herein, refers to any and/or all forms of coconut oil, including fractionated and unrefined, as defined herein.

“Moisturizer,” as used herein, refers to any lipid-based agent suitable for topical application with emollient properties, other than coconut oil.

The terms “active ingredient” and “active agent,” as used herein, refer to ingredients that produce a natural, homeopathic, medicinal, pharmaceutical or cosmetic effect. In the present disclosure, coconut oil is an active agent. The term “additional active agent” or “additional active ingredient” refers to any active agent or ingredient other than coconut oil.

The terms “zinc,” “elemental zinc,” and/or “ionic zinc,” refer to zinc in an ionic form that is bioavailable and may be suitable for topical application to the body.

The term “carrier,” as used herein, refers to any solvent used in conjunction with the active agent including, but not limited to, aqueous, alcoholic, and oleaginous solvents.

The term “nasal cavity,” as used herein, refers to all aspects of the nasal cavity, including the nostrils, nasal/mucus membrane, cilia, and sinuses.

The term “effective,” as used herein, refers to achieving desired effects and/or alleviating adverse effects upon administration to the body of at least one active agent.

### DETAILED DESCRIPTION OF THE INVENTION

The present disclosure relates to natural and homeopathic compositions comprising coconut oil as an active agent and exhibiting moisturizing and antimicrobial properties, particularly antiviral properties, when applied topically. The composition of the present invention may also be medicinal, pharmaceutical, and/or cosmetic compositions.

In all aspects of the present disclosure, coconut oil is present as an active agent. In one aspect of the present disclosure, coconut oil comprises an amount ranging from 0.1 -99.9% by weight of the composition, for example from 10-90% by weight, 20-80% by weight, 30-60% by weight, and even from 40-50% by weight of the composition. In some aspects of the present disclosure, the composition comprises a mixture of unrefined coconut oil and refined coconut oil.

In one aspect of the present disclosure, unrefined coconut oil comprises an amount ranging from 0.1-99.9% by weight of the composition, for example 20-80% by weight, 20-60% by weight, 30-50% by weight, and even from 40-50% by weight of the composition. Non-limiting examples of unrefined coconut oil include coconut oil 76 and coconut oil 92, such as those oils produced and sold by Oils by Nature.

In another aspect of the present disclosure, fractionated coconut oil comprises an amount ranging from 0.1-99.9% by weight of the composition, for example from 0.1-20% by weight, 0.1-5% by weight, or even 0.5-2% by weight of the composition. As a non-limiting example, fractionated coconut oil includes those oils produced and sold by Oils by Nature.

In at least one aspect of the present disclosure, the compositions may also comprise at least one moisturizer other than coconut oil. According to the present disclosure, the at least one moisturizer may be present in an amount ranging from 0.1-99.9% by weight of the composition, for example from 20-80% by weight, 40-80% by weight, 40-70% by weight, and even 40-60% by weight of the composition. Non-limiting examples of the at least one moisturizer include shea butter, cosmetic grade shea butter, glycerol/glycerine, aloe vera, and combinations thereof.

In at least one embodiment, the present disclosure relates to a natural and/or homeopathic composition for topical application to the nasal cavity or lips comprising 40% by weight unrefined coconut oil and 60% by weight cosmetic grade shea butter. In at least one embodiment of the present disclosure said composition is a cream, lotion, or solid. In another embodiment of the present disclosure, the natural and/or homeopathic composition is in a form suitable for application to the lips and is effective in preventing and/or treating the herpes virus.

The present disclosure further relates to natural and/or homeopathic compositions comprising coconut oil as an active agent and at least one additional active agent, wherein the composition is intended for topical application. The at least one additional active agent may impart natural, homeopathic, medicinal, pharmaceutical, and/or cosmetic properties to the composition.

Non-limiting examples of the at least one additional active agent of the present disclosure include trace minerals such as zinc, manganese, and selenium, decongestants, antihistamines, and any other natural, homeopathic, medicinal, pharmaceutical, and/or cosmetic product that may be combined with coconut oil in a form suitable for topical administration. The at least one additional active agent may be present in an amount ranging from 0.1-99.9% by weight of the composition, for example from 0.1-50% by weight, 0.1-25% by weight, 0.1-10% by weight, 0.1-5% by weight, or even 0.1-0.9% by weight of the composition.

More specifically, one aspect of the present disclosure relates to compositions comprising zinc as the at least one additional active agent. Said additional active agent is in the form of a zinc salt that provides a source of elemental or ionic zinc for application to the body. Non-limiting examples of zinc for use in the present disclosure include zinc acetate, zinc chloride, zinc gluconate, zinc oxide, and zinc sulfate. In at least one aspect of the present disclosure, at least one zinc salt may be present in an amount such that the amount of zinc present in the composition ranges from 0.1-10% by weight, 0.1-5% by weight, or even 0.1-0.9% by weight of the composition.

Other aspects of the present disclosure relate to compositions comprising other trace minerals as the at least one additional active agent. In at least one embodiment, the at least one additional active agent comprises manganese. In other embodiments, the at least one additional active agent comprises selenium.

In other aspects of the present disclosure, the composition further comprises at least one carrier. Non-limiting examples of the at least one carrier include aqueous solvents such as distilled water, alcoholic solvents such as ethanol, oleaginous solvents, and mixtures thereof. The at least one carrier may be used in conjunction with the at least one additional active ingredient. The at least one carrier may be present in an amount ranging from 0.1-99.9% by weight, for example 0.1-50% by weight, 0.1-25% by weight, 0.1-10% by weight, or even 0.1-5% by weight of the composition.

In at least one embodiment of the present disclosure, distilled water is the at least one carrier. In at least one other embodiment of the present disclosure, the distilled water is present in an amount ranging from 0.1-50% by weight of the composition.

In another embodiment of the present disclosure, zinc sulfate is the at least one additional active agent and the at least one carrier is distilled water. In another embodiment of the present disclosure, zinc gluconate is the at least one additional active agent and the at least one carrier is distilled water. In at least one further embodiment, zinc salt is dissolved in distilled water before addition to the oily phase during preparation of the composition.

The compositions of the present disclosure may also contain other pharmaceutically and/or cosmetically acceptable adjuvants, including, but not limited to, fillers, surfactants, dyestuff, preservatives, emulsifying agents, stabilizers, antioxidants, moisturizers, and other adjuvants known and used in the art.

One of ordinary skill in the art would recognize the amount of coconut oil, any additional active agent, moisturizer, carrier, and/or adjuvant presently disclosed may vary depending on the composition and choice of at least one additional active agent. The skilled artisan would thus take care to select the particular components and concentrations thereof to achieve advantageous properties associated with the compositions in an acceptable form, whether a natural, homeopathic, medicinal, pharmaceutical, and/or cosmetic composition.

The compositions presently disclosed may be in a form suitable for topical application. Non-limiting examples of the forms of the presently disclosed compositions include emulsions, suspensions, creams, lotions, gels, liquids, solids, and sprays. In one embodiment, the composition of the present disclosure is formulated for application to the nasal cavity, such as in the form of a lotion, cream, or gel. In another embodiment, the composition is used in combination with an applicator. In another embodiment, the composition is formulated for application to the lips, such as in a lip balm or solid lip product.

In some embodiments of the present disclosure, the viscosity of the composition ranges from about 500 to 3000 centipoise at 25° C. In at least one embodiment, the viscosity of the composition is greater than or equal to about 1500 centipoise at 25° C. For example, in some embodiments, the viscosity may range from about 1500 to 3000 centipoise at 25° C., such as about 1500 to 2500 centipoise at 25° C.

In another aspect of the present disclosure, the viscosity of the composition is less than 1500 centipoise at 25° C. In at least one embodiment, the viscosity of the composition ranges from 700 to 1499 centipoise at 25° C., for example from 700 to 1250 centipoise at 25° C., and also about 750 to 1000 centipoise at 25° C. The viscosities of the compositions disclosed herein may be measured with a viscometer suitable for making said measurement at 25° C., for example a Brookfield viscometer Model RVT.

The present disclosure further relates to methods of using these compositions to promote good health and/or for treatment and/or prevention of disease.

One aspect of the present disclosure is a method for promoting good health and/or for treatment and/or prevention of disease, comprising application to the body of a natural and/or homeopathic composition comprising coconut oil as described herein.

At least one embodiment of the present disclosure is a method for promoting good health and/or treating and/or preventing disease comprising topical administration of a composition comprising 40-80% by weight unrefined coconut oil and 20-60% by weight cosmetic grade shea butter. In some embodiments, the composition may further comprise at least one other pharmaceutically and/or cosmetically acceptable adjuvant to add up to 100%. In at least one embodiment, the composition comprises 40-80% by weight unrefined coconut oil, 20-60% by weight cosmetic grade shea butter, and 0.1% to 10% by weight of at least one other pharmaceutically and/or cosmetically acceptable adjuvant. At least one other embodiment of the present disclosure relates to application of the composition to the nasal cavity. Yet another embodiment of the present disclosure relates to application of the composition to the lips.

Another aspect of the present disclosure is a method for promoting good health and/or for treatment and/or prevention of disease, comprising application to the body of a natural and/or homeopathic composition comprising coconut oil and at least one additional active agent as described herein. In a further aspect of the disclosure, the at least one additional active agent imparts natural, homeopathic, medicinal, pharmaceutical, and/or cosmetic properties to the composition.

At least one embodiment of the present disclosure is a method for treating and/or preventing the common cold in a subject, including alleviating at least one symptom associated with the common cold, comprising application to the subject of a composition comprising 40-50% by weight of at least one unrefined coconut oil; 0.5-2% by weight of at least one fractionated coconut oil; 40-50% by weight of at least one moisturizer; 0.1-0.9% zinc sulfate or zinc gluconate (at least one additional active agent); and 0.1-10% distilled water. In one embodiment of the present disclosure, the composition is applied to the nasal cavity of a subject for treating and/or preventing the common cold.

Unless expressly noted to the contrary, all compositions described herein are expressed as weight percent based on the total weight of the composition.

Other than in the examples, or where otherwise indicated, all numbers expressing quantities of ingredients, reaction conditions, analytical measurements, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples that follow are intended to illustrate the present disclosure without, however, being limiting in nature.

### EXAMPLES

The present disclosure may be understood more readily by reference to the following detailed description of exemplary embodiments and the working examples. It is understood that other embodiments will become apparent to those skilled in the art in view of the description and examples disclosed in this specification.

### Example 1

A composition in accordance with the present disclosure was prepared as follows:

Coconut oil 92 (40 g) was heated to 70° C. and liquefied. In a separate container, shea butter (60 g) was heated to 70° C. and liquefied. The coconut oil and shea butter were then combined and mixed at 70° C. with stirring for a minimum of 1 hour. The mixture was removed from heat and allowed to cool slowly to room temperature under stirring. Stirring was stopped when the mixture began to congeal. Before the mixture hardened, it was poured into applicator tubes. The tubes were sealed, labeled, and stored at room temperature.

### Example 2

A composition in accordance with the present disclosure was prepared for application in the nasal cavity according to the following:

Shea butter (45 g), coconut oil fractionated (1 g), and coconut oil 76 (46.8 g) were combined and warmed to 60° C. with stirring until the components were evenly liquefied and dispersed. In a separate beaker, distilled water (5 g) was warmed to 60° C. and zinc sulfate heptahydrate (2.2 g) added with stirring until completely dissolved. The zinc mixture was slowly added to the oil mixture and stirred at EMP (electric mortar and pestle) setting 2:00/5, allowed to rest 20 minutes, and then stirred again at setting 2:00/0.

The mixture was slowly cooled, allowed to rest for at least 24 hours, and stored at room temperature (25° C.). The mixture was then warmed to 70° C. and poured into an applicator tube. The tube was sealed, labeled, and stored at room temperature.

The resulting composition viscosity was measured at 821 centipoise at 25° C. by a Brookfield Model RVT viscometer.

### Example 3

The composition prepared according to Example 2 is administered to the nasal cavity of a human subject exhibiting cold-like symptoms for less than 24 hours. The subject reports nasal congestion, headache, muscle/body ache and sore throat. Prior to application at room temperature (25° C.), the composition has the consistency of a cream or lotion. Upon contact with the nasal membrane of the subject, the cream melts into a liquid oily phase creating a barrier on the nasal membrane while active ingredients pass through the subject's nasal membrane. The subject reports reduced nasal congestion, sore throat, headache, and muscle/body ache.

### Example 4

The compositions prepared according to Example 1 and Example 2 were submitted for in-vitro testing to assess and compare their antiviral activities against coronaviruses.

Viral agents and associated host cells were obtained from the American Type Culture Collection (ATCC). Feline coronavirus (Feline Infectious Peritonitis Virus, FIPV, ATCC VR-2201) was propagated on monolayers of A-72 canine tumor fibroblast cells (ATCC CRL-1542) grown in L-15 medium supplemented with 10% fetal calf serum. Human coronavirus (HCV) strain 229 (ATCC VR-740) was cultured in human lung fibroblast cells (MRC5, ATCC CRL-171) grown in EMEM with 10% fetal calf serum. Host cell stocks were passaged weekly in T-162 flasks, incubated at 37° C. under 5% CO₂. Virus stocks were prepared in T-162 flasks containing host cells at 85% confluence. Host monolayers were inoculated with 1-2 ml of the appropriate virus, and the flasks were incubated at 37° C. for 1 hr to facilitate virus attachment. Maintenance medium (growth medium supplemented with 2% fetal calf serum) was then added and flasks returned to the incubator for 5-7 days and observed daily for cytopathic effects (host cell rounding and sloughing). Medium containing free virus and infected cells was harvested, and cells fractured by three rounds of freeze-thawing. Lysates were collected in 5 ml aliquots and maintained at −72° C.

All virus assays were conducted in 24-well tissue culture plates (Costar). Appropriate host cells were inoculated into the plates and grown to 85% confluence. Virus-containing samples were serially diluted (10-fold) in maintenance medium, and 0.1 ml volumes inoculated onto quadruplicate wells. Following a 1 hr adsorption period (37° C., 5% CO₂), 1 ml of maintenance medium was added to each well and the plates returned to the incubator. Wells were observed for CPE over a period of either 1 week (FIPV) or 2 weeks (HCV). Tissue culture infective dose (TCID50) was calculated according to the method of Reed and Munch (1938).

One hundred mg amounts of each of the Example 1 and Example 2 compositions were melted in a water bath (60° C.) and mixed with 10 ml of 60° C. tissue culture growth medium (10% fetal calf serum). The test product was then serially diluted through similarly heated medium to obtain the desired working concentrations of coconut oil or zinc sulfate. Each stock was then filter sterilized (0.22 um) and cooled to 37° C. One milliliter volumes were mixed with equal volumes of test virus and incubated for 1 hr at 37° C. Samples were then serially diluted and assayed as described above.

Experimental controls were set up as follows:

Negative controls consisted of wells containing only host cells and medium.

For virus controls, virus stock was mixed 1:2 with growth medium, incubated at 37° C. for 1 hr, serially diluted and assayed. This assay provided data on initial (i.e. untreated) virus concentrations used in the experiment.

For toxicity control, the filtered product stock solutions were mixed with equal volumes of growth medium, incubated for 1 hr at 37° C., and serially diluted in maintenance medium. Volumes of 0.1 ml were inoculated onto quadruplicate wells containing host cells, incubated for 1 hr and each well supplemented with growth medium and returned to the incubator. This control provided data on the relative toxicity of the product for the host cells.

The antiviral activity of the two test products is shown in Table 1. Both FIPV AND HCV virus types demonstrated sensitivity to fairly low doses of coconut oil. The lowest tested concentration affecting FIPV inactivation was 25 μg/ml, while HCV inactivation appeared to begin at 2.5 μg/ml. Table 1 shows treatment with a composition of Example 1 having a coconut oil concentration of 2500 μg/ml yielded significantly less viral inactivation than treatment with a composition of Example 1 having a coconut oil concentration of 1200 μg/ml. Likewise, treatment with a composition of Example 2 having a coconut oil concentration of 2390 μg/ml showed less viral inactivation than treatment with a composition of Example 2 having a coconut oil concentration of 1195 μg/ml.

TABLE 1
Coronavirus inactivation by coconut oil containing products

| | | | Concentration of | Virus titer | |
|---|---|---|---|---|---|
| | | Active | active ingredient | reduction | % Viral |
| Virus | Composition | Ingredient | (ug/ml) | (log₁₀) | inactivation |
| FIPV | Example 1 | Coconut oil | 0.25 | NR* | 0 |
| FIPV | Example 1 | Coconut oil | 2.5 | NR | 0 |
| FIPV | Example 1 | Coconut oil | 25 | 0.4 | 60 |
| FIPV | Example 1 | Coconut oil | 250 | 0.7 | 80 |
| FIPV | Example 1 | Coconut oil | 500 | 1.0 | 90 |
| FIPV | Example 1 | Coconut oil | 625 | 0.8 | 84 |
| FIPV | Example 1 | Coconut oil | 833 | 1.0 | 90 |
| FIPV | Example 1 | Coconut oil | 1200 | 1.8 | 98.4 |
| FIPV | Example 1 | Coconut oil | 2500** | 0.3 | 49.3 |
| HCV | Example 1 | Coconut oil | 0.025 | NR | 0 |
| HCV | Example 1 | Coconut oil | 0.25 | NR | 0 |
| HCV | Example 1 | Coconut oil | 2.5 | 0.2 | 36.7 |
| HCV | Example 1 | Coconut oil | 25 | 0.8 | 84.2 |
| FIPV | Example 2 | Coconut oil/Zn | 23.9/0.25 | 0.5 | 68.7 |
| FIPV | Example 2 | Coconut oil/Zn | 239/2.5 | 0.7 | 80 |
| FIPV | Example 2 | Coconut oil/Zn | 1195/12.5 | 0.9 | 87.5 |
| FIPV | Example 2 | Coconut oil/Zn | 2390/25** | 0.3 | 49.3 |

*no reduction
**probable loss of product during filtration

### Example 5

Open label clinical studies are performed to test antiviral activity of the Example 1 composition on human subjects. The composition is administered to subjects exposed to the common cold who are asked to report the severity and duration of their symptoms. Independent research boards (IRB) are employed to assess the activity of the composition regarding prevention and/or duration of infection, and the severity of the subjects' symptoms.

We claim:

1. A natural and/or homeopathic composition comprising:
at least one coconut oil; and
at least one moisturizer;
wherein said composition is acceptable for topical administration.

2. The composition of claim 1, wherein the at least one coconut oil comprises unrefined coconut oil in an amount ranging from 20-60 weight percent.

3. The composition of claim 1, wherein said at least one coconut oil comprises unrefined coconut oil in an amount ranging from about 40-50 weight percent and further comprises fractionated coconut oil in an amount ranging from about 0.5-2 weight percent.

4. The composition according to claim 1, wherein the at least one moisturizer comprises shea butter in an amount ranging from 40-80 weight percent.

5. The composition according to claim 1, further comprising at least one additional active agent.

6. The composition according to claim 5, wherein the at least one additional active agent comprises a trace mineral chosen from zinc, manganese, and selenium.

7. The composition according to claim 6, wherein the at least one additional active agent comprising zinc is chosen from zinc acetate, zinc chloride, zinc gluconate, zinc oxide, and zinc sulfate.

8. The composition according to claim 1, wherein the composition is in a form acceptable for application to the nasal cavity or lips.

9. The composition according to claim 1, wherein the composition is in the form of an emulsion, suspension, cream, lotion, gel, liquid, or spray.

10. The composition according to claim 1, wherein the viscosity of the composition ranges from about 500 to 3000 centipoise at 25° C.

11. The composition according to claim 1, wherein the viscosity of the composition is less than 1500 centipoise at 25° C.

12. The composition according to claim 11, wherein the viscosity of the composition ranges from 750 to 1000 centipoise at 25° C.

13. A natural and/or homeopathic composition comprising:
40-50 weight percent of at least one unrefined coconut oil;
0.5-2 weight percent of at least one fractionated coconut oil;
40-50 weight percent of at least one moisturizer;
0.1-25 weight percent of at least one additional active agent; and
0.1-10 weight percent of at least one carrier;
wherein said composition is acceptable for topical administration.

14. The composition of claim 13, wherein the at least one additional active agent is present in an amount ranging from 0.1-0.9 weight percent.

15. The composition of claim 14, wherein the at least one additional active agent is chosen from zinc sulfate and zinc gluconate.

16. A method for treating and/or preventing disease comprising application of at least one natural and/or homeopathic composition comprising:
at least one coconut oil; and
at least one moisturizer.

17. The method of claim 16, wherein said composition is applied to the nasal cavity or lips.

18. The method of claim 16, wherein said composition comprises at least one additional active agent.

19. A method of alleviating at least one symptom associated with the common cold in a subject comprising topically applying a natural and/or homeopathic composition comprising:
40-50 weight percent of at least one unrefined coconut oil;
0.5-2 weight percent of at least one fractionated coconut oil;
40-50 weight percent of at least one moisturizer;
0.1-0.9 weight percent of at least one additional active agent; and
0.1-10 weight percent of at least one carrier.

20. The method of claim 19, wherein the at least one additional active agent is chosen from zinc sulfate and zinc gluconate.
